(19) United States
(12) Patent Application Publication
(10) Pub. No.: US 2010/0316589 A1
(43) Pub. Date: Dec. 16, 2010

(54) Coated Pharmaceutical Compositions

(76) Inventors: Hitesh Bhagat, Wayland, MA (US); Adnan Salameh, Irvine, CA (US)

Correspondence Address: Genzyme Corporation, 153 Second Avenue, Waltham, MA, 02451, US

(21) Appl. No.: 12/808,039

(22) PCT Filed: Dec. 12, 2008
(86) PCT No.: PCT/US08/13675

§ 371 Date: Sep. 2, 2010

Related U.S. Application Data
(60) Provisional application No. 61/006,020, filed on Dec. 14, 2007.

(51) Int. Cl. A61K 31/785; A61P 3/00; A61P 13/12

(52) U.S. Cl. 424/78.17

(57) ABSTRACT

The present invention relates to polycarbophil coated crosslinked amine polymers and/or pharmaceutical compositions comprising polycarbophil coated crosslinked amine polymers. The polycarbophil coated crosslinked amine polymers have several therapeutic applications, including, but not limited to, hyperphosphatemia, chronic kidney disease and End-Stage Renal Disease.

### CROSS REFERENCE TO RELATED APPLICATION

This application claims the benefit of U.S. Provisional Application No. 61/006,020, filed Dec. 14, 2008, and is incorporated herein by reference in its entirety.

### FIELD OF THE INVENTION

This invention relates to pharmaceutically acceptable compositions and polymers or residues thereof for binding target ions, and more specifically relates to polycarbophil coated polymer particles for binding target ions.

### BACKGROUND OF THE INVENTION

Hyperphosphatemia frequently accompanies diseases associated with inadequate renal function such as end stage renal disease (ESRD), hyperparathyroidism, and certain other medical conditions. The condition, especially if present over extended periods of time, leads to severe abnormalities in calcium and phosphorus metabolism and can be manifested by aberrant calcification in joints, lungs, and eyes.

Therapeutic efforts to reduce serum phosphate include dialysis, reduction in dietary phosphate, and oral administration of insoluble phosphate binders to reduce gastrointestinal absorption. Many such treatments have a variety of unwanted side effects and/or have less than optimal phosphate binding properties, including potency and efficacy. Accordingly, there is a need for compositions and treatments with good phosphate-binding properties and good side effect profiles.

### BRIEF SUMMARY OF THE INVENTION

In one aspect, the present invention relates to polycarbophil coated crosslinked amine polymers and/or pharmaceutical compositions comprising, at least in part, polycarbophil coated crosslinked amine polymers. Compositions can comprise one or more polycarbophil coated crosslinked amine polymers. Several embodiments of the invention are described in further detail as follows. Generally, each of these embodiments can be used in various and specific combinations, and with other aspects and embodiments unless otherwise stated herein.

In addition to the polycarbophil coated crosslinked amine polymers of the present invention as described herein, other forms of the polycarbophil coated crosslinked amine polymers are within the scope of the invention including pharmaceutically acceptable salts, solvates, hydrates, prodrugs, polymorphs, clathrates, and isotopic variants and mixtures thereof of the polycarbophil coated crosslinked amine polymers.

In addition, polycarbophil coated crosslinked amine polymers of the invention may have optical centers, chiral centers or double bonds and the polycarbophil coated crosslinked amine polymers of the present invention include all of the isomeric forms of these polycarbophil coated crosslinked amine polymers, including optically pure forms, racemates, diastereomers, enantiomers, tautomers and/or mixtures thereof.

In some embodiments, the polycarbophil coated crosslinked amine polymer is in the form of crosslinked amine polymer particles coated with polycarbophil. In some embodiments, the polycarbophil coated crosslinked amine polymer particles have an in-vitro competitive phosphate binding capacity that is between 2 and 10 times the in-vitro competitive phosphate binding capacity of uncoated epichlorohydrin-crosslinked polyallylamine hydrochloride. In some embodiments, the polycarbophil coated crosslinked amine polymer particles have an in vitro non-competitive phosphate binding capacity that is substantially the same as epichlorohydrin-crosslinked polyallylamine hydrochloride. In some embodiments, the polycarbophil coated crosslinked amine polymer particles have a change in in vitro competitive phosphate binding capacity between 1 and 5 hours of less than 50%. In some embodiments, the polycarbophil coated crosslinked amine polymer particles have an in vitro competitive phosphate binding capacity at 1 hour of greater than 0.5 mmol/g. In some embodiments, the polycarbophil coated crosslinked amine polymer particles have an in vitro competitive phosphate binding capacity at 5 hours of greater than 0.5 mmol/g.

In some embodiments, the polycarbophil coated crosslinked amine polymer particles comprise a coating that further includes N-(3-dimethylaminopropyl)-N′-ethylcarbodiimide hydrochloride. In some embodiments, the polycarbophil coated crosslinked amine polymer particles comprise a coating that comprises a thermally-treated coating. In some embodiments, the polycarbophil coated crosslinked amine polymer particles comprise a coating that comprises an ionically coupled coating. In some embodiments, the polycarbophil coated crosslinked amine polymer particles comprise a coating that has a coating stability at pH 1 such that less than 50% of said coating is lost. In some embodiments, the polycarbophil coated crosslinked amine polymer particles comprise a coating that has a coating stability in the presence of counterions wherein 70% or less of said coating is lost.

In some embodiments, the invention provides methods of treating an animal, including a human. The method generally involves administering an effective amount of one or more polycarbophil coated crosslinked amine polymers or a composition (e.g., a pharmaceutical composition) comprising the same to the animal as described herein.

In some embodiments, the invention is, consists essentially of, or comprises a polycarbophil coated crosslinked amine polymer, a pharmaceutical composition comprising a polycarbophil coated crosslinked amine polymer or a method for removing a compound or ion, such as a phosphorous-containing compound or a phosphorous-containing ion (e.g., phosphate), from the gastrointestinal tract of an animal by administering an effective amount of a polycarbophil coated crosslinked amine polymer or a pharmaceutical composition comprising a polycarbophil coated crosslinked amine polymer, wherein the crosslinked amine polymer includes repeat units represented by the following Formula I:

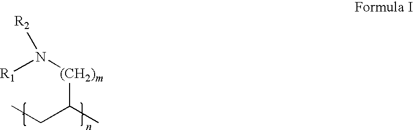

or a copolymer thereof, wherein m is an integer from 0 to 2, n is an integer and each R₁ and each R₂ independently represent hydrogen; substituted or unsubstituted, branched or unbranched C₁-C₆ alkyl; or substituted or unsubstituted, branched or unbranched C₁-C₆ alkylamino.

In some embodiments, the invention is, consists essentially of, or comprises a polycarbophil coated crosslinked amine polymer, a pharmaceutical composition comprising a polycarbophil coated crosslinked amine polymer or a method for removing a compound or ion, such as a phosphorous-containing compound or a phosphorous-containing ion (e.g., phosphate), from the gastrointestinal tract of an animal by administering an effective amount of a polycarbophil coated crosslinked amine polymer or a pharmaceutical composition comprising a polycarbophil coated crosslinked amine polymer, wherein the crosslinked amine polymer includes repeat units represented by the following Formula II:

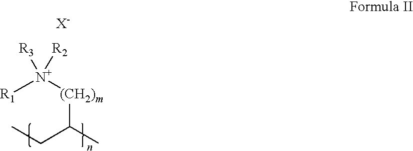

or a copolymer thereof, wherein m is an integer from 0 to 2, n is an integer each R₁ and each R₂ independently represent hydrogen; substituted or unsubstituted, branched or unbranched C₁-C₆ alkyl; or substituted or unsubstituted, branched or unbranched C₁-C₆ alkylamino; and each X⁻ independently represents a pharmaceutically acceptable counterion.

In some embodiments, the invention is, consists essentially of, or comprises a polycarbophil coated crosslinked amine polymer, a pharmaceutical composition comprising a polycarbophil coated crosslinked amine polymer or a method for removing a compound or ion, such as a phosphorous-containing compound or a phosphorous-containing ion (e.g., phosphate), from the gastrointestinal tract of an animal by administering an effective amount of a polycarbophil coated crosslinked amine polymer or a pharmaceutical composition comprising a polycarbophil coated crosslinked amine polymer, wherein the crosslinked amine polymer includes repeat units represented by the following Formula III:

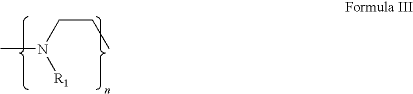

or a copolymer thereof, wherein n is an integer and each R₁ and each R₂ independently represent hydrogen; substituted or unsubstituted, branched or unbranched C₁-C₆ alkyl; or substituted or unsubstituted, branched or unbranched C₁-C₆ alkylamino.

In some embodiments, the invention is, consists essentially of, or comprises a polycarbophil coated crosslinked amine polymer, a pharmaceutical composition comprising a polycarbophil coated crosslinked amine polymer or a method for removing a compound or ion, such as a phosphorous-containing compound or a phosphorous-containing ion (e.g., phosphate), from the gastrointestinal tract of an animal by administering an effective amount of a polycarbophil coated crosslinked amine polymer or a pharmaceutical composition comprising a polycarbophil coated crosslinked amine polymer, wherein the crosslinked amine polymer includes repeat units represented by the following Formula IV:

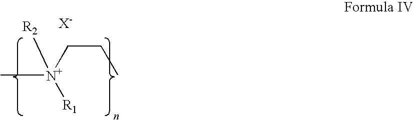

or a copolymer thereof, wherein n is an integer and each R₁ and each R₂ independently represent hydrogen; substituted or unsubstituted, branched or unbranched C₁-C₆ alkyl; or substituted or unsubstituted, branched or unbranched C₁-C₆ alkylamino; and each X⁻ independently represents a pharmaceutically acceptable counterion.

In some embodiments, the invention is, consists essentially of, or comprises a polycarbophil coated crosslinked amine polymer comprising substituted or unsubstituted polyallylamine crosslinked with epichlorohydrin, wherein the crosslinked amine polymer is coated with polycarbophil.

Another aspect of the invention is a pharmaceutical composition comprising one or more polycarbophil coated crosslinked amine polymers and at least one pharmaceutically acceptable excipient. The polycarbophil coated crosslinked amine polymers described herein have several therapeutic applications. For example, the polycarbophil coated crosslinked amine polymers are useful in removing compounds or ions such as anions, for example phosphorous-containing compounds or phosphorous containing ions such as organophosphates and/or phosphates, from the gastrointestinal tract, such as from the stomach, small intestine and/or large intestine. In some embodiments, the polycarbophil coated crosslinked amine polymers are used in the treatment of phosphate imbalance disorders and renal diseases.

In yet another aspect, the polycarbophil coated crosslinked amine polymers are useful for removing other solutes, such as chloride, bicarbonate, and/or oxalate containing compounds or ions. Polycarbophil coated crosslinked amine polymers removing oxalate compounds or ions find use in the treatment of oxalate imbalance disorders. Polycarbophil coated crosslinked amine polymers removing chloride compounds or ions find use in treating acidosis, for example. In some embodiments, the polycarbophil coated crosslinked amine polymers are useful for removing bile acids, citrate and related compounds.

In some embodiments, the polycarbophil coated crosslinked amine polymers may be in the form of a composition that is a liquid formulation in which the polycarbophil coated crosslinked amine polymer is dispersed in a liquid vehicle, such as water, and suitable excipients. In some embodiments, the invention provides a pharmaceutical composition comprising a polycarbophil coated crosslinked amine polymer for binding a target compound or ion, and one or more suitable pharmaceutical excipients, where the composition is in the form of a tablet, sachet, slurry, food formulation, troche, capsule, elixir, suspension, syrup, wafer, chewing gum or lozenge. In some embodiments the composition contains a pharmaceutical excipient selected from the group consisting of sucrose, mannitol, xylitol, maltodextrin, fructose, sorbitol, and combinations thereof. In some embodiments the target anion of the polycarbophil coated crosslinked amine polymer is an organophosphate and/or phosphate. In some embodiments the polycarbophil coated crosslinked amine polymer is more than about 50% of the weight of the tablet. In some embodiments, the tablet is of cylindrical shape with a diameter of from about 12 mm to about 28 mm and a height of from about 1 mm to about 8 mm and the polycarbophil crosslinked amine polymer comprises more than 0.6 to about 2.0 gm of the total weight of the tablet.

In some of the compositions of the invention, the excipients are chosen from the group consisting of sweetening agents, binders, lubricants, flavoring agents and disintegrants. In some of these embodiments, the sweetening agent is selected from the group consisting of sucrose, mannitol, xylitol, maltodextrin, fructose, and sorbitol, and combinations thereof.

### DETAILED DESCRIPTION OF THE INVENTION

In one aspect, the present invention provides polycarbophil coated crosslinked amine polymers, compositions and methods of using polycarbophil coated crosslinked amine polymers, where the crosslinked amine polymer is represented by repeat units according to any of Formulas I-IV. In addition, some embodiments may include multiple different repeat units or residues thereof that repeat in a copolymer or polymer. Such polymers or copolymers may include one or more additional compounds that may be included in a polymer backbone or as pendant groups either individually or as repeating groups.

As used herein, unless otherwise stated, the term “derived from” is understood to mean: produced or obtained from another substance by chemical reaction, especially directly derived from the reactants, for example a crosslinked amine polymer may be derived from the reaction of an amine monomer or amine polymer and a linking agent, such as a crosslinking agent resulting in a crosslinked amine polymer that is derived from the amine monomer or amine polymer and the crosslinking agent.

In some embodiments, the invention is, consists essentially of, or comprises a polycarbophil coated crosslinked amine polymer, a pharmaceutical composition comprising a polycarbophil coated crosslinked amine polymer or a method for removing a compound or ion, such as a phosphorous-containing compound or a phosphorous-containing ion (e.g., phosphate), from the gastrointestinal tract of an animal by administering an effective amount of a polycarbophil coated crosslinked amine polymer or a pharmaceutical composition comprising a polycarbophil coated crosslinked amine polymer, wherein the crosslinked amine polymer comprises repeat units represented by the following Formula I:

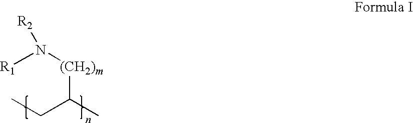

or a copolymer thereof, wherein m is an integer from 0 to 2, such as for example, 0, 1 or 2; n is an integer and each R₁ and each R₂ independently represent hydrogen; substituted or unsubstituted, branched or unbranched C₁-C₆ alkyl, such as C₁, C₂, C₃, C₄, C₅ or C₆ alkyl; substituted or unsubstituted, branched or unbranched C₁-C₆ alkylamino such as C₁, C₂, C₃, C₄, C₅ or C₆ alkylamino; or a link, such as a crosslink or other link.

Examples of some suitable crosslinked amine polymer repeat units according to Formula I include:

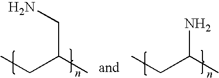

In some embodiments, the invention is, consists essentially of, or comprises a polycarbophil coated crosslinked amine polymer, a pharmaceutical composition comprising a polycarbophil coated crosslinked amine polymer or a method for removing a compound or ion, such as a phosphorous-containing compound or a phosphorous-containing ion (e.g., phosphate), from the gastrointestinal tract of an animal by administering an effective amount of a polycarbophil coated crosslinked amine polymer or a pharmaceutical composition comprising a polycarbophil coated crosslinked amine polymer wherein the crosslinked amine polymer comprises repeat units represented by the following Formula II:

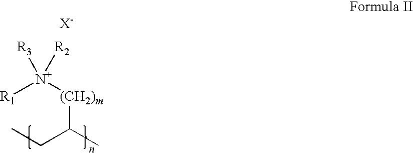

or a copolymer thereof, wherein m is an integer from 0 to 2, such as for example, 0, 1 or 2; n is an integer and each R₁ and each R₂ independently represent hydrogen; substituted or unsubstituted, branched or unbranched C₁-C₆ alkyl, such as C₁, C₂, C₃, C₄, C₅ or C₆ alkyl; substituted or unsubstituted, branched or unbranched C₁-C₆ alkylamino such as C₁, C₂, C₃, C₄, C₅ or C₆ alkylamino; or a link, such as a crosslink or other link; and each X⁻ independently represents a pharmaceutically acceptable counterion.

In some embodiments, the invention is, consists essentially of, or comprises a polycarbophil coated crosslinked amine polymer, a pharmaceutical composition comprising a polycarbophil coated crosslinked amine polymer or a method for removing a compound or ion, such as a phosphorous-containing compound or a phosphorous-containing ion (e.g., phosphate), from the gastrointestinal tract of an animal by administering an effective amount of a polycarbophil coated crosslinked amine polymer or a pharmaceutical composition comprising a polycarbophil coated crosslinked amine polymer wherein the crosslinked amine polymer comprises repeat units represented by the following Formula III:

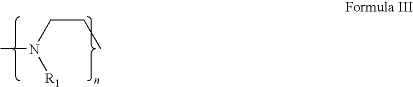

or a copolymer thereof, wherein n is an integer and each R₁ independently represent hydrogen; substituted or unsubstituted, branched or unbranched C₁-C₆ alkyl, such as C₁, C₂, C₃, C₄, C₅ or C₆ alkyl; substituted or unsubstituted, branched or unbranched C₁-C₆ alkylamino such as C₁, C₂, C₃, C₄, C₅ or C₆ alkylamino; or a link, such as a crosslink or other link.

In some embodiments, the invention is, consists essentially of, or comprises a polycarbophil coated crosslinked amine polymer, a pharmaceutical composition comprising a polycarbophil coated crosslinked amine polymer or a method for removing a compound or ion, such as a phosphorous-containing compound or a phosphorous-containing ion (e.g., phosphate), from the gastrointestinal tract of an animal by administering an effective amount of a polycarbophil coated crosslinked amine polymer or a pharmaceutical composition comprising a polycarbophil coated crosslinked amine polymer wherein the crosslinked amine polymer comprises repeat units represented by the following Formula IV:

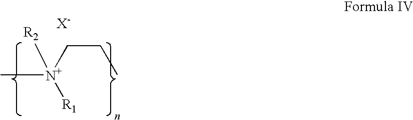

or a copolymer thereof, wherein n is an integer and each R₁ and each R₂ independently represent hydrogen; substituted or unsubstituted, branched or unbranched C₁-C₆ alkyl, such as C₁, C₂, C₃, C₄, C₅ or C₆ alkyl; substituted or unsubstituted, branched or unbranched C₁-C₆ alkylamino such as C₁, C₂, C₃, C₄, C₅ or C₆ alkylamino; or a link, such as a crosslink or other link; and each X⁻ independently represents a pharmaceutically acceptable counterion.

In some embodiments, the invention is a method of treating a phosphate imbalance disorder such as hyperphosphatemia comprising administering a therapeutically effective amount of one or more polycarbophil coated crosslinked amine polymers or copolymers of the invention or a composition comprising one or more one or more polycarbophil coated crosslinked amine polymers or copolymers of the invention to a patient in need thereof.

In some embodiments, the composition includes a mixture of more than one polycarbophil coated crosslinked amine polymers or copolymer, for example 2-20, such as 2, 3, 4, 5, 6, 7, 8, 9 or 10, polycarbophil coated crosslinked amine polymers or copolymers.

In some embodiments, the invention is, consists essentially of, or comprises a polycarbophil coated crosslinked amine polymer, a pharmaceutical composition comprising a polycarbophil coated crosslinked amine polymer or a method for removing a compound or ion, such as a phosphorous-containing compound or a phosphorous-containing ion (e.g., phosphate), from the gastrointestinal tract of an animal by administering an effective amount of a polycarbophil coated crosslinked amine polymer or a pharmaceutical composition comprising a polycarbophil coated crosslinked amine polymer wherein the crosslinked amine polymer is derived from: a monomer selected from substituted or unsubstituted allylamine and substituted or unsubstituted ethyleneimine; and a crosslinking agent.

In some embodiments, the invention is, consists essentially of, or comprises a polycarbophil coated crosslinked amine polymer, a pharmaceutical composition comprising a polycarbophil coated crosslinked amine polymer or a method for removing a compound or ion, such as a phosphorous-containing compound or a phosphorous-containing ion (phosphate), from the gastrointestinal tract of an animal by administering an effective amount of a polycarbophil coated crosslinked amine polymer or a pharmaceutical composition comprising a polycarbophil coated crosslinked amine polymer wherein the crosslinked amine polymer is derived from: a monomer selected from substituted or unsubstituted allylamine and epichlorohydrin as a crosslinking agent.

In some embodiments, the invention is, consists essentially of, or comprises a polycarbophil coated crosslinked amine polymer, a pharmaceutical composition comprising a polycarbophil coated crosslinked amine polymer or a method for removing a compound or ion, such as a phosphorous-containing compound or a phosphorous-containing ion (e.g., phosphate), from the gastrointestinal tract of an animal by administering an effective amount of a polycarbophil coated crosslinked amine polymer or a pharmaceutical composition comprising a polycarbophil coated crosslinked amine polymer, where the crosslinked amine polymer comprises polyallylamine crosslinked with epichlorohydrin.

In some embodiments, the invention is, consists essentially of, or comprises a polycarbophil coated crosslinked amine polymer, a pharmaceutical composition comprising a polycarbophil coated crosslinked amine polymer or a method for removing a compound or ion, such as a phosphorous-containing compound or a phosphorous-containing ion (e.g., phosphate), from the gastrointestinal tract of an animal by administering an effective amount of a polycarbophil coated crosslinked amine polymer or a pharmaceutical composition comprising a polycarbophil coated crosslinked amine polymer, where the crosslinked amine polymer comprises polyallylamine crosslinked with epichlorohydrin and is partially or fully protonated with a pharmaceutically acceptable counterion as the counterion.

In some embodiments, the invention is, consists essentially of, or comprises a polycarbophil coated crosslinked amine polymer, a pharmaceutical composition comprising a polycarbophil coated crosslinked amine polymer or a method for removing a compound or ion, such as a phosphorous-containing compound or a phosphorous-containing ion (phosphate), from the gastrointestinal tract of an animal by administering an effective amount of a polycarbophil coated crosslinked amine polymer or a pharmaceutical composition comprising a polycarbophil coated crosslinked amine polymer, where the crosslinked amine polymer comprises polyallylamine crosslinked with epichlorohydrin and is partially or fully protonated with carbonate or chloride as the counterion.

In some embodiments, the invention is a method for reducing blood phosphate levels by at least 0.1 mg/dl to 5 mg/dl, such as by at least 0.2 mg/dl, at least 0.4 mg/dl, at least 0.8 mg/dl, at least 1.0 mg/dl, at least 1.5 mg/dl, at least 2.0 mg/dl, at least 2.5 mg/dl, at least 3.0 mg/dl, at least 3.5 mg/dl, at least 4.0 mg/dl or by at least 4.5 mg/dl in a patient in need thereof, the method comprising administering a therapeutically effective amount of a polycarbophil coated crosslinked amine polymer or composition comprising the same to the patient. In some embodiments, the invention is a method for reducing urinary phosphorous by at least 0.1 mg/dl to 5 mg/dl, such as by at least 0.2 mg/dl, at least 0.4 mg/dl, at least 0.8 mg/dl, at least 1.0 mg/dl, at least 1.5 mg/dl, at least 2.0 mg/dl, at least 2.5 mg/dl, at least 3.0 mg/dl, at least 3.5 mg/dl, at least 4.0 mg/dl or by at least 4.5 mg/dl in a patient in need thereof, the method comprising administering a therapeutically effective amount of a polycarbophil coated crosslinked amine polymer or composition comprising the same to the patient.

### Polymerization

In some embodiments, prior to coating, the amine polymers may be crosslinked in a solution of bulk (i.e. using the neat amine polymer and neat crosslinking agents) or in dispersed media. When a bulk process is used, solvents are selected so that they co-dissolve the reactants and do not interfere with the crosslinking reaction. Suitable solvents include water, low boiling alcohols (methanol, ethanol, butanol), dimethylformamide, dimethylsulfoxide, acetone, acetonitrile, methylethylketone, and the like.

Other polymerization methods may include a single polymerization reaction, stepwise addition of individual monomers via a series of reactions, the stepwise addition of blocks of monomers, combinations of the foregoing, or any other method of polymerization, such as, for example, direct or inverse suspension, condensation, phase transfer, emulsion, precipitation techniques, polymerization in aerosol or using bulk polymerization/crosslinking methods and other control processes such as extrusion and grinding. Processes can be carried out as batch, semi-continuous and continuous processes. For processes in dispersed media, the continuous phase can be selected from apolar solvents such as toluene, benzene, hydrocarbon, halogenated solvents, supercritical carbon dioxide, and the like. With a direct suspension process, water can be used, although salt brines are also useful to “salt out” the amine and crosslinking agents in a droplet separate phase.

A non-limiting example of polymerization of polyallylamine with epichlorohydrin may occur as follows. Polyallylamine hydrochloride in water may be partially neutralized using a base such as ammonium hydroxide (aqueous ammonia) or NaOH. After neutralization, the polyallylamine may be emulsified with epichlorohydrin using a static or high shear mixer. The resulting oil in water emulsion may be polymerized using a single or twin screw kneading or LIST reactor. The polymer leaving the LIST reactor may be washed multiple times and protonated using a suitable source such as HCl, CO₂ or carbonic acid, and may be milled and/or separated before drying using any suitable technique such as centrifugal force, such as using hydrocyclones or centrifuges. The polymer may also be dried using a convection oven, a vacuum oven or a fluidized bed and then may be sieved after drying.

Examples of other suitable polymerization methods may be found, for example, in the following patents and patent applications each of which is incorporated herein by reference in their entirety: U.S. Pat. No. 4,605,701; U.S. Pat. No. 5,496,545; U.S. Pat. No. 5,618,530; U.S. Pat. No. 5,679,717; U.S. Pat. No. 5,693,675; U.S. Pat. No. 5,702,696; US WO 96/21454; WO 98/57652; EP 7372352; and DE 4227019.

Before coating, the crosslinked amine polymers may be copolymers that may comprise a monomer comprising a compound having at least one unit according to any of Formulas I-IV which is copolymerized with one or more other comonomers or oligomers or other polymerizable groups.

Non-limiting examples of suitable comonomers which may be used alone or in combination include: styrene, substituted styrene, alkyl acrylate, substituted alkyl acrylate, alkyl methacrylate, substituted alkyl methacrylate, acrylonitrile, methacrylonitrile, acrylamide, methacrylamide, N-alkylacrylamide, N-alkylmethacrylamide, N,N-dialkylacrylamide, N,N-dialkylmethacrylamide, isoprene, butadiene, ethylene, vinyl acetate, N-vinyl amide, maleic acid derivatives, vinyl ether, allyle, methallyl monomers and combinations thereof. Functionalized versions of these monomers may also be used. Additional specific monomers or comonomers that may be used in this invention include, but are not limited to, methyl methacrylate, ethyl methacrylate, propyl methacrylate (all isomers), butyl methacrylate (all isomers), 2-ethylhexyl methacrylate, isobornyl methacrylate, methacrylic acid, benzyl methacrylate, phenyl methacrylate, methyl acrylate, ethyl acrylate, propyl acrylate (all isomers), butyl acrylate (all isomers), 2-ethylhexyl acrylate, isobornyl acrylate, acrylic acid, benzyl acrylate, phenyl acrylate, acrylonitrile, styrene, N,N-dimethylaminoethyl methacrylate, N,N-diethylaminoethyl methacrylate, N,N-dimethylaminoethyl acrylate, N,N-diethylaminoethyl acrylate, maleic anhydride, allylamine, methallylamine, allylalcohol, butadiene, isoprene, chloroprene, ethylene, vinyl acetate and combinations thereof.

In addition, the polycarbophil coated crosslinked amine polymers of the invention may comprise copolymers having any combination of repeat units according to Formulas I-IV. For example, in some embodiments, the crosslinked amine polymers comprise a combination of repeat units according to Formulas I and II, Formulas I and III, Formulas I and IV, Formulas II and III, Formulas II and IV, Formulas III and IV, Formulas I, II and III, Formulas I, II and IV, Formulas I, III and IV, Formulas II, III and IV or Formulas I, II, III and IV.

In some embodiments, prior to coating, the crosslinked amine polymers of the invention may not dissolve in solvents, and, at most, swell in solvents The swelling ratio may be calculated according to the procedure in the Test Methods section below and is typically in the range of about 5 to about 150, such as 5 to about 100, 5 to about 80, 5 to about 60, 5 to about 40, or 5 to about 20; for example, 5 to 18, 5 to 16 or 5 to 15, such as greater than 5 and less than 40, greater than 5 and less than 20, greater than 9 and less than 20, greater than 11 and less than 20, such as 5, 6, 7, 8, 9, 10, 11, 12, 13, 14, 15, 16, 17, 18, 19 or more.

Crosslinking agents are typically compounds having at least two functional groups that are selected from a halogen group, carbonyl group, epoxy group, ester group, acid anhydride group, acid halide group, isocyanate group, vinyl group, and chloroformate group. The crosslinking agent may be attached to the carbon backbone or to a nitrogen of an amine polymer, amine monomer or residue thereof.

Examples of crosslinking agents that are suitable for synthesis of the crosslinked amine polymers include, but are not limited to, one or more multifunctional crosslinking agents such as: dihaloalkanes, haloalkyloxiranes, alkyloxirane sulfonates, di(haloalkyl)amines, tri(haloalkyl)amines, diepoxides, triepoxides, tetraepoxides, bis(halomethyl)benzenes, tri(halomethyl)benzenes, tetra(halomethyl)benzenes, epihalohydrins such as epichlorohydrin and epibromohydrin, poly(epichlorohydrin), (iodomethyl)oxirane, bromo-1,2-epoxybutane, 1,2-dibromoethane, 1,3-dichloropropane, 1,2-dichloroethane, 1-bromo-2-chloroethane, 1,3-dibromopropane, bis(2-chloroethyl)amine, tris(2-chloroethyl)amine, and bis(2-chloroethyl)methylamine, 1,3-butadiene diepoxide, 1,5-hexadiene diepoxide, methyl acrylate and the like. When the crosslinking agent is an alkylhalide compound, a base may be used to scavenge the acid formed during the reaction. When used, inorganic or organic bases are suitable with NaOH being preferred. The base to crosslinking agent ratio may be between about 0.5 to about 2.

In some embodiments, the crosslinking agents may be used in the crosslinking reaction in an amount of from 0.5 mol % to 30 mol %, based on the amount of amine repeat unit, such as from about 5 mol % to about 15 mol %, from about 7 mol % to about 12 mol %, from about 8.5 mol % to about 10.5 mol %, from about 9 mol % to about 10 mol %, such as 2, 3, 4, 5, 6, 7, 8, 9, 9.4, 9.8, 10, 11, 12, 13, 14, 15, 16, 17, 18, 19, 20, 22, 25, 27 or 30 mol %.

In some embodiments, the weight averaged molecular weight of the amine polymers and copolymers, prior to crosslinking and coating, may be typically at least about 1000. For example, the molecular weight may be from about 1000 to about 1,000,000, such as about 2000 to about 750,000, about 3000 to about 500,000, about 5000 to about 250,000, about 10000 to about 100,000, such as from 15,000-80,000, 20,000 to 75,000, 25,000 to 60,000, 30,000 to 50,000, or 40,000 to 45,000.

The crosslinked amine polymers of some embodiments may be formed using a polymerization initiator. Generally, any initiator may be used including cationic and radical initiators. Some examples of suitable initiators that may be used include: the free radical peroxy and azo type compounds, such as azodiisobutyronitrile, azodiisovaleronitrile, dimethylazodiisobutyrate, 2,2′-azobis(isobutyronitrile), 2,2′-azobis(N,N′-dimethyleneisobutyramidine)dihydrochloride, 2,2′-azobis(2-amidinopropane)dihydrochloride, 2,2′-azobis(N,N′-dimethyleneisobutyramidine), 1,1′-azobis(1-cyclohexanecarbo-nitrile), 4,4′-azobis(4-cyanopentanoic acid), 2,2′-azobis(isobutyramide) dihydrate, 2,2′-azobis(2-methylpropane), 2,2′-azobis(2-methylbutyronitrile), VAZO 67, cyanopentanoic acid, the peroxy pivalates, dodecylbenzene peroxide, benzoyl peroxide, di-t-butyl hydroperoxide, t-butyl peracetate, acetyl peroxide, dicumyl peroxide, cumyl hydroperoxide, dimethyl bis(butylperoxy)hexane.

In some embodiments, any of the nitrogen atoms within the crosslinked amine polymers, prior to coating, may optionally be quaternized to yield the corresponding positively charged tertiary nitrogen group, such as for example, an ammonium or substituted ammonium group. Any one or more of the nitrogen atoms in the crosslinked amine polymers may be quaternized and such quaternization, when present, is not limited to or required to include terminal amine nitrogen atoms. In some embodiments, this quaternization may result in additional network formation and may be the result of addition of crosslinking, linking or amine reactive groups to the nitrogen. The ammonium groups may be associated with a pharmaceutically acceptable counterion.

In some embodiments, prior to coating, the crosslinked amine polymers may be partially or fully quaternized, including protonated, with a pharmaceutically acceptable counterion, which may be organic ions, inorganic ions, or a combination thereof. Examples of some suitable inorganic ions include halides (e.g., chloride, bromide or iodide) carbonates, bicarbonates, sulfates, bisulfates, hydroxides, nitrates, persulfates and sulfites. Examples of some suitable organic ions include acetates, ascorbates, benzoates, citrates, dihydrogen citrates, hydrogen citrates, oxalates, succinates, tartrates, taurocholates, glycocholates, and cholates. Preferred counterions include chlorides and carbonates.

In some embodiments, prior to coating, crosslinked amine polymers may be protonated such that the fraction of protonated nitrogen atoms is from 1% to 100%, such as 10% to 75%, 20% to 60%, 25%% to 55%, 30% to 50%, 35% to 45% or about 40%.

In one embodiment, prior to coating, a pharmaceutically acceptable crosslinked amine polymer is in protonated form and comprises a carbonate anion. In one embodiment, prior to coating, the pharmaceutically acceptable crosslinked amine polymer is in protonated form and comprises a mixture of carbonate and bicarbonate counterions.

### Coating

The crosslinked amine polymers may be coated with polycarbophil. In some embodiments, particles of the crosslinked amine polymers may be ionically coated with polycarbophil. In some embodiments, ionic coating is achieved by mixing the particles of a cationic crosslinked amine polymer with polycarbophil in the presence of a solvent, such as water. The resulting coated particles may then be filtered and dried. In some embodiments, the dry ionically coated particles may be thermally treated by placing them in an oven or by using any other thermal treatment method. In some embodiments, such thermal treatment may be conducted in a vacuum oven at 80 to 120° C., such as 85° C., 90° C., 95° C., 100° C., 105° C., 110° C. or 115° C. and may last 5 hours or more, such as 10 hours or more, 12 hours or more, 14 hours or more, 16 hours or more, 18 hours or more, 20 hours or more, 24 hours or more.

In other embodiments, particles of the crosslinked amine polymers may be coated by chemically coupling them to the polycarbophil using a coupling agent. In some embodiments, the coupling agent may comprise N-(3-dimethylaminopropyl)-N′-ethylcarbodiimide hydrochloride. Chemical coupling may achieved by mixing the polycarbophil with the chemical coupling agent and then adding this mixture to a suspension of the crosslinked amine polymer in water. The resulting coated particles may then be filtered and dried. In some embodiments, the dry chemically coupled polycarbophil coated particles may be thermally treated by placing them in an oven or by using any other thermal treatment method. In some embodiments, such thermal treatment may be conducted in a vacuum oven at 80 to 120° C., such as 85° C., 90° C., 95° C., 100° C., 105° C., 110° C. or 115° C. and may last 5 hours or more, such as 10 hours or more, 12 hours or more, 14 hours or more, 16 hours or more, 18 hours or more, 20 hours or more, 24 hours or more.

In some embodiments, the coating can occur in-situ. In such embodiments, particles of the crosslinked amine polymers may be blended with polycarbophil using any suitable blending equipment and then formed into the dosage form such that the coating forms in-situ.

Any other suitable coating methods may be used to coat the crosslinked amine polymer particles with polycarbophil. The polycarbophil used in the coating may be any suitable grade of polycarbophil, such as USP grade polycarbophil or polycarbophil made in accordance with a USP monograph. The polycarbophil used in the coating may be an acrylic acid polymer, such as a homopolymer, that is crosslinked with divinyl glycol. In some embodiments, the polycarbophil may comprise a high molecular weight acrylic acid polymer, such as a homopolymer, that is crosslinked with divinyl glycol. In some embodiments, the polycarbophil may be in the form of a salt, such as a calcium salt of such divinyl glycol crosslinked acrylic acid polymers. In some embodiments, the polycarbophil may be coarse milled or finely milled.

In some embodiments, polycarbophil coated crosslinked amine polymers of the invention have a coating stability such that 60% or less, such as 50% or less, 45% or less, 40% or less, 35% or less, 30% or less, 25% or less, 20% or less, 15% or less or 10% or less of said coating is lost at pH 1. In some embodiments, polycarbophil coated crosslinked amine polymers of the invention have a coating stability such that 40% or less, such as 30% or less, 25% or less, 20% or less, 15% or less, 10% or less or 7.5% or less of said coating is lost at pH 4. In some embodiments, polycarbophil coated crosslinked amine polymers of the invention have a coating stability such that 40% or less, such as 30% or less, 25% or less, 20% or less, 15% or less, 10% or less or 7.5% or less of said coating is lost at pH 6. In some embodiments, polycarbophil coated crosslinked amine polymers of the invention have a coating stability such that 40% or less, such as 30% or less, 25% or less, 20% or less, 15% or less, 10% or less or 7.5% or less of said coating is lost at pH 8.

In some embodiments, polycarbophil coated crosslinked amine polymers of the invention have a coating stability such that 70% or less, such as 60% or less, 50% or less, 40% or less, 35% or less, 30% or less, 25% or less, 20% or less, 15% or less, 10% or less or 1% or less in the presence of counterions.

In some embodiments, polycarbophil coated crosslinked amine polymers of the invention are characterized by their ability to bind compounds or ions. Preferably the polycarbophil coated crosslinked amine polymers of the invention bind anions, more preferably they bind suitable organophosphates, phosphate and/or oxalate, and most preferably they bind organophosphates or phosphate. For illustration, anion-binding polycarbophil coated crosslinked amine polymers and especially organophosphate or phosphate-binding polycarbophil coated crosslinked amine polymers will be described; however, it is understood that this description applies equally, with appropriate modifications that will be apparent to those of skill in the art, to other ions, compounds and solutes. Polycarbophil coated crosslinked amine polymers may bind an ion, e.g., an anion, when they associate with the ion, generally though not necessarily in a noncovalent manner, with sufficient association strength that at least a portion of the ion remains bound under the in vitro or in vivo conditions in which the polymer is used for sufficient time to effect a removal of the ion from solution or from the body. A target ion may be an ion to which the polycarbophil coated crosslinked amine polymer binds, and usually refers to the ion whose binding to the polycarbophil coated crosslinked amine polymer is thought to produce the therapeutic effect of the polymer and may be an anion or a cation. A polycarbophil coated crosslinked amine polymer of the invention may have more than one target ion.

In some embodiments, the polycarbophil coated crosslinked amine polymer particles have an in-vitro competitive phosphate binding capacity that is between 2 and 10 times, such as between 3 and 9 times, between 4 and 8 times, between 5 and 7 times, between 2 and 5 times, between 3 and 5 times, between 3 and 4 times or between 3 and 6 times the in-vitro competitive phosphate binding capacity of uncoated epichlorohydrin-crosslinked polyallylamine hydrochloride. The in vitro competitive phosphate binding capacity may be measured according to the techniques described in the Test Methods section below.

In some embodiments, the polycarbophil coated crosslinked amine polymer particles have an in vitro non-competitive phosphate binding capacity that is substantially the same as uncoated epichlorohydrin-crosslinked polyallylamine hydrochloride. In some embodiments, the in vitro non-competitive phosphate binding capacity of the polycarbophil coated crosslinked amine polymer particles is greater than about 0.2, 0.4, 0.5, 1.0, 1.5, 2.0, 2.5, 3.0, 3.5, 4.0, 4.5, 5.0, 6.0, 8.0, 10.0, 12, or greater than 14 mmol/g. In some embodiments, the in vitro non-competitive phosphate binding capacity of polycarbophil coated crosslinked amine polymers of the invention is greater than about 0.4 mmol/g, greater than about 2.5 mmol/g, greater than about 3 mmol/g, greater than about 4.5 mmol/g or greater than about 6 mmol/g. In some embodiments, the in vitro non-competitive phosphate binding capacity of polycarbophil coated crosslinked amine polymers of the invention can be between about 0.2 mmol/g and about 14 mmol/g, such as between about 0.4 mmol/g and about 10 mmol/g, between about 2.5 mmol/g and about 8 mmol/g or between about 3 mmol/g and about 6 mmol/g. The in vitro non-competitive phosphate binding capacity may be measured according to the techniques described in the Test Methods section below.

In some embodiments, the polycarbophil coated crosslinked amine polymer particles have an in vitro competitive phosphate binding capacity at 1 hour of between 0.5 mmol/g and 10 mmol/g, such as between 0.75 mmol/g and 9 mmol/g, between 1.0 mmol/g and 8.5 mmol/g, between 1.25 mmol/g and 8.0 mmol/g, between 1.5 mmol/g and 7.5 mmol/g, between 1.75 mmol/g and 7.0 mmol/g, between 2.0 mmol/g and 6.5 mmol/g, between 2.5 mmol/g and 6.0 mmol/g, between 2.75 mmol/g and 5.5 mmol/g, between 3 mmol/g and 5.0 mmol/g at 1 hour and/or an in vitro competitive phosphate binding capacity at 5 hours of between 0.5 mmol/g and 10 mmol/g, such as between 0.75 mmol/g and 9 mmol/g, between 1.0 mmol/g and 8.5 mmol/g, between 1.25 mmol/g and 8.0 mmol/g, between 1.5 mmol/g and 7.5 mmol/g, between 1.75 mmol/g and 7.0 mmol/g, between 2.0 mmol/g and 6.5 mmol/g, between 2.5 mmol/g and 6.0 mmol/g, between 2.75 mmol/g and 5.5 mmol/g, between 3 mmol/g and 5.0 mmol/g at 5 hours. The in vitro competitive phosphate binding capacity may be measured according to the techniques described in the Test Methods section below.

In some embodiments, the polycarbophil coated crosslinked amine polymer particles have a change in in vitro competitive phosphate binding capacity between 1 and 5 hours of less than 50%, such as less than 45%, less than 40%, less than 35%, less than 30%, less than 25%, less than 20%, less than 15%, less than 10% or less than 5%. The in vitro competitive phosphate binding capacity may be measured according to the techniques described in the Test Methods section below.

In some embodiments, the polycarbophil coated crosslinked amine polymers of the present invention have an in vitro competitive phosphate binding capacity at 5 hours of greater than 20%, for example greater than 30%, greater than 35%, greater than 40%, greater than 45%, greater than 50%, greater than 60%, greater than 70% or greater than 80% of the 1 hour in vitro competitive phosphate binding capacity of said polymer. The in vitro competitive phosphate binding capacity may be measured according to the techniques described in the Test Methods section below.

In some embodiments, the polycarbophil coated crosslinked amine polymers according to the invention have an in vitro competitive phosphate binding capacity of between 0.5 mmol/g and 10 mmol/g, such as between 0.75 mmol/g and 9 mmol/g, between 1.0 mmol/g and 8.5 mmol/g, between 1.25 mmol/g and 8.0 mmol/g, between 1.5 mmol/g and 7.5 mmol/g, between 1.75 mmol/g and 7.0 mmol/g, between 2.0 mmol/g and 6.5 mmol/g, between 2.5 mmol/g and 6.0 mmol/g, between 2.75 mmol/g and 5.5 mmol/g, between 3 mmol/g and 5.0 mmol/g throughout a physiologically significant time period. A physiologically significant time period may be the length of time during which significant uptake of a target ion occurs in a human. For example, for phosphate the physiologically significant time period may be from 0 to 5 hours, such as 0.5 to 5 hours, 1 to 5 hours, 1 to 4.5 hours, 1.5 to 4 hours, 2 to 3.5 hours or for greater than 1, 1.5, 2, 2.5, 3, 3.5, 4, 4.5 or 5 hours. The in vitro competitive phosphate binding capacity may be measured according to the techniques described in the Test Methods section below.

In some embodiments, the polycarbophil coated crosslinked amine polymers of the present invention have an in vitro non-competitive phosphate binding capacity at 5 hours that is within 20%, for example within 15%, 12.5%, 10% or even 5% of that of sevelamer hydrochloride. The in vitro non-competitive phosphate binding capacity may be measured according to the techniques described in the Test Methods section below.

In some embodiments, the polycarbophil coated crosslinked amine polymers of the present invention have an in vitro competitive phosphate binding capacity at 1 hour of greater than 20%, for example greater than 30%, greater than 35%, greater than 40%, greater than 45%, greater than 50%, greater than 60%, greater than 70% or greater than 80% of the 5 hour in vitro non-competitive phosphate binding capacity of said polymer. The in vitro non-competitive phosphate binding capacity and the in vitro competitive phosphate binding capacity may be measured according to the techniques described in the Test Methods section below.

In some embodiments, the polycarbophil coated crosslinked amine polymers of the present invention have an in vitro competitive phosphate binding capacity at 5 hours of greater than 20%, for example greater than 30%, greater than 35%, greater than 40%, greater than 45%, greater than 50%, greater than 60%, greater than 70% or greater than 80% of the 5 hour in vitro non-competitive phosphate binding capacity of said polymer. The in vitro competitive phosphate binding capacity and the in vitro competitive phosphate binding capacity may be measured according to the techniques described in the Test Methods section below.

In some embodiments, the polycarbophil coated crosslinked amine polymers of the invention have an in vivo phosphate binding capacity of between 0.2 mmol/g and 14 mmol/g, such as between 0.3 mmol/g and 13 mmol/g, between 0.4 mmol/g and 12.5 mmol/g, between 0.5 mmol/g and 10 mmol/g, between 0.75 mmol/g and 8 mmol/g, between 1.0 mmol/g and 6 mmol/g, between 1.25 mmol/g and 5 mmol/g, between 1.5 mmol/g and 4.5 mmol/g, between 2.0 mmol/g and 4.0 mmol/g or between 2.5 mmol/g and 3.5 mmol/g. The in vivo phosphate binding capacity may be measured in any animal, such as any mammal, such as humans or rats. The test methods detail a procedure for measuring the in vivo phosphate binding capacity in rats, which may be suitably modified as appropriate for measurement in humans.

In some embodiments, the polycarbophil coated crosslinked amine polymers of the invention have an in vitro bile acid binding capacity of between 0.5 mmol/g and 5.0 mmol/g, such as between 1.0 mmol/g and 5 mmol/g, between 1.5 mmol/g and 4.5 mmol/g, between 2.0 mmol/g and 4.0 mmol/g, between 2.25 mmol/g and 4.0 mmol/g, between 2.5 mmol/g and 4.0 mmol/g, such as greater than 1.00, 1.25, 1.5, 1.75, 2.0, 2.25, 2.5, 2.75, 3.0, 3.25, 3.5, 3.75, 4, 4.25, 4.5 or greater than 4.75 mmol/g. The in vitro bile acid binding capacity may be determined according to the procedure detailed in the Test Procedures.

In some embodiments, the polycarbophil coated crosslinked amine polymer comprises a repeat unit according to Formula I, where m is 0 or 1, n is an integer, R₁ and R₂ are H or a link, where the polymer is crosslinked with 9.8 mol % or 10 mol % epichlorohydrin crosslinker, where the crosslinked amine polymer is in the form of a base or a hydrochloride or carbonate salt and particles that are ionically coated with polycarbophil and where the polycarbophil coated crosslinked amine polymer has: an in vitro competitive phosphate binding capacity that is 2 to 10 times that of uncoated epichlorohydrin crosslinked polyallylamine hydrochloride, a change in in vitro competitive phosphate binding capacity between 1 and 5 hours of less than 50%, an in vitro competitive phosphate binding capacity at 1 hour of greater than 0.5 mmol/g, an in vitro competitive phosphate binding capacity at 5 hours of greater than 0.5 mmol/g, a coating that has a coating stability at pH 1 such that less than 50% of said coating is lost, a coating that has a coating stability at pH 4 such that less than 30% of said coating is lost and/or a coating that has a coating stability in the presence of counterions wherein 70% or less of said coating is lost.

In other embodiments, the polycarbophil coated crosslinked amine polymer comprises a repeat unit according to Formula I, where m is 0 or 1, n is an integer, R₁ and R₂ are H or a link, where the polymer is crosslinked with 9.8 mol % or 10 mol % epichlorohydrin crosslinker, where the crosslinked amine polymer is in the form of a base or a hydrochloride or carbonate salt and particles that are chemically coated with polycarbophil using EDC as a coupling agent and where the polycarbophil coated crosslinked amine polymer has: an in vitro competitive phosphate binding capacity that is 2 to 10 times that of uncoated epichlorohydrin crosslinked polyallylamine hydrochloride, a change in in vitro competitive phosphate binding capacity between 1 and 5 hours of less than 50%, an in vitro competitive phosphate binding capacity at 1 hour of greater than 0.5 mmol/g, an in vitro competitive phosphate binding capacity at 5 hours of greater than 0.5 mmol/g, a coating that has a coating stability at pH 1 such that less than 50% of said coating is lost, a coating that has a coating stability at pH 4 such that less than 30% of said coating is lost and/or a coating that has a coating stability in the presence of counterions wherein 70% or less of said coating is lost.

In some embodiments, the polycarbophil coated crosslinked amine polymer comprises a repeat unit according to Formula I, where m is 0 or 1, n is an integer, R₁ and R₂ are H or a link, where the polymer is crosslinked with 9.8 mol % or 10 mol % epichlorohydrin crosslinker, where the crosslinked amine polymer is in the form of a base or a hydrochloride or carbonate salt and particles that are ionically coated or chemically coated with polycarbophil where the coating is thermally treated and where the polycarbophil coated crosslinked amine polymer has: an in vitro competitive phosphate binding capacity that is 2 to 10 times that of uncoated epichlorohydrin crosslinked polyallylamine hydrochloride, a change in in vitro competitive phosphate binding capacity between 1 and 5 hours of less than 50%, an in vitro competitive phosphate binding capacity at 1 hour of greater than 0.5 mmol/g, an in vitro competitive phosphate binding capacity at 5 hours of greater than 0.5 mmol/g, a coating that has a coating stability at pH 1 such that less than 50% of said coating is lost, a coating that has a coating stability at pH 4 such that less than 30% of said coating is lost and/or a coating that has a coating stability in the presence of counterions wherein 70% or less of said coating is lost.

In some embodiments, the polycarbophil coated crosslinked amine polymer comprises a repeat unit according to Formula I, where m is 0 or 1, n is an integer, R₁ and R₂ are H or a link, where the polymer is crosslinked with 15 mol % or 20 mol % epichlorohydrin crosslinker, where the crosslinked amine polymer is in the form of a base or a hydrochloride or carbonate salt and particles that are ionically coated with polycarbophil and where the polycarbophil coated crosslinked amine polymer has: an in vitro competitive phosphate binding capacity that is 2 to 10 times that of uncoated epichlorohydrin crosslinked polyallylamine hydrochloride, a change in in vitro competitive phosphate binding capacity between 1 and 5 hours of less than 50%, an in vitro competitive phosphate binding capacity at 1 hour of greater than 0.5 mmol/g, an in vitro competitive phosphate binding capacity at 5 hours of greater than 0.5 mmol/g, a coating that has a coating stability at pH 1 such that less than 50% of said coating is lost, a coating that has a coating stability at pH 4 such that less than 30% of said coating is lost and/or a coating that has a coating stability in the presence of counterions wherein 70% or less of said coating is lost.

In other embodiments, the polycarbophil coated crosslinked amine polymer comprises a repeat unit according to Formula I, where m is 0 or 1, n is an integer, R₁ and R₂ are H or a link, where the polymer is crosslinked with 15 mol % or 20 mol % epichlorohydrin crosslinker, where the crosslinked amine polymer is in the form of a base or a hydrochloride or carbonate salt and particles that are chemically coated with polycarbophil using EDC as a coupling agent and where the polycarbophil coated crosslinked amine polymer has: an in vitro competitive phosphate binding capacity that is 2 to 10 times that of uncoated epichlorohydrin crosslinked polyallylamine hydrochloride, a change in in vitro competitive phosphate binding capacity between 1 and 5 hours of less than 50%, an in vitro competitive phosphate binding capacity at 1 hour of greater than 0.5 mmol/g, an in vitro competitive phosphate binding capacity at 5 hours of greater than 0.5 mmol/g, a coating that has a coating stability at pH 1 such that less than 50% of said coating is lost, a coating that has a coating stability at pH 4 such that less than 30% of said coating is lost and/or a coating that has a coating stability in the presence of counterions wherein 70% or less of said coating is lost.

In some embodiments, the polycarbophil coated crosslinked amine polymer comprises a repeat unit according to Formula I, where m is 0 or 1, n is an integer, R₁ and R₂ are H or a link, where the polymer is crosslinked with 15 mol % or 20 mol % epichlorohydrin crosslinker, where the crosslinked amine polymer is in the form of a base or a hydrochloride or carbonate salt and particles that are ionically coated or chemically coated with polycarbophil where the coating is thermally treated and where the polycarbophil coated crosslinked amine polymer has: an in vitro competitive phosphate binding capacity that is 2 to 10 times that of uncoated epichlorohydrin crosslinked polyallylamine hydrochloride, a change in in vitro competitive phosphate binding capacity between 1 and 5 hours of less than 50%, an in vitro competitive phosphate binding capacity at 1 hour of greater than 0.5 mmol/g, an in vitro competitive phosphate binding capacity at 5 hours of greater than 0.5 mmol/g, a coating that has a coating stability at pH 1 such that less than 50% of said coating is lost, a coating that has a coating stability at pH 4 such that less than 30% of said coating is lost and/or a coating that has a coating stability in the presence of counterions wherein 70% or less of said coating is lost.

In some embodiments, the polycarbophil coated crosslinked amine polymer comprises polyallylamine crosslinked with epichlorohydrin and then coated with polycarbophil. In some embodiments, the crosslinked amine polymer comprises polyallylamine crosslinked with 9.8 mol % or 10 mol % epichlorohydrin, where the polymer is in the form of a base or a hydrochloride or carbonate salt and particles that are ionically coated with polycarbophil and where the polycarbophil coated crosslinked amine polymer has: an in vitro competitive phosphate binding capacity that is 2 to 10 times that of uncoated epichlorohydrin crosslinked polyallylamine hydrochloride, a change in in vitro competitive phosphate binding capacity between 1 and 5 hours of less than 50%, an in vitro competitive phosphate binding capacity at 1 hour of greater than 0.5 mmol/g, an in vitro competitive phosphate binding capacity at 5 hours of greater than 0.5 mmol/g, a coating that has a coating stability at pH 1 such that less than 50% of said coating is lost, a coating that has a coating stability at pH 4 such that less than 30% of said coating is lost and/or a coating that has a coating stability in the presence of counterions wherein 70% or less of said coating is lost.

In some embodiments, the crosslinked amine polymer comprises polyallylamine crosslinked with 9.8 mol % or 10 mol % epichlorohydrin, where the polymer is in the form of a base or a hydrochloride or carbonate salt and particles that are chemically coated with polycarbophil using EDC as a coupling agent and where the polycarbophil coated crosslinked amine polymer has: an in vitro competitive phosphate binding capacity that is 2 to 10 times that of uncoated epichlorohydrin crosslinked polyallylamine hydrochloride, a change in in vitro competitive phosphate binding capacity between 1 and 5 hours of less than 50%, an in vitro competitive phosphate binding capacity at 1 hour of greater than 0.5 mmol/g, an in vitro competitive phosphate binding capacity at 5 hours of greater than 0.5 mmol/g, a coating that has a coating stability at pH 1 such that less than 50% of said coating is lost, a coating that has a coating stability at pH 4 such that less than 30% of said coating is lost and/or a coating that has a coating stability in the presence of counterions wherein 70% or less of said coating is lost.

In some embodiments, the crosslinked amine polymer comprises polyallylamine crosslinked with 9.8 mol % or 10 mol % epichlorohydrin, where the polymer is in the form of a base or a hydrochloride or carbonate salt and particles that are ionically coated or chemically coated with polycarbophil, where the coating is thermally treated and where the polycarbophil coated crosslinked amine polymer has: an in vitro competitive phosphate binding capacity that is 2 to 10 times that of uncoated epichlorohydrin crosslinked polyallylamine hydrochloride, a change in in vitro competitive phosphate binding capacity between 1 and 5 hours of less than 50%, an in vitro competitive phosphate binding capacity at 1 hour of greater than 0.5 mmol/g, an in vitro competitive phosphate binding capacity at 5 hours of greater than 0.5 mmol/g, a coating that has a coating stability at pH 1 such that less than 50% of said coating is lost, a coating that has a coating stability at pH 4 such that less than 30% of said coating is lost and/or a coating that has a coating stability in the presence of counterions wherein 70% or less of said coating is lost.

In some embodiments, the polycarbophil coated crosslinked amine polymer comprises a repeat unit according to the following formula:

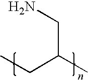

which is crosslinked with 9.8 mol % or 10 mol % epichlorohydrin, where the crosslinked amine polymer is in the form of a base or a hydrochloride or carbonate salt and particles that are ionically coated with polycarbophil and where the polycarbophil coated crosslinked amine polymer has: an in vitro competitive phosphate binding capacity that is 2 to 10 times that of uncoated epichlorohydrin crosslinked polyallylamine hydrochloride, a change in in vitro competitive phosphate binding capacity between 1 and 5 hours of less than 50%, an in vitro competitive phosphate binding capacity at 1 hour of greater than 0.5 mmol/g, an in vitro competitive phosphate binding capacity at 5 hours of greater than 0.5 mmol/g, a coating that has a coating stability at pH 1 such that less than 50% of said coating is lost, a coating that has a coating stability at pH 4 such that less than 30% of said coating is lost and/or a coating that has a coating stability in the presence of counterions wherein 70% or less of said coating is lost.

In some embodiments, the polycarbophil coated crosslinked amine polymer comprises a repeat unit according to the following formula:

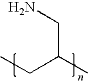

which is crosslinked with 9.8 mol % or 10 mol % epichlorohydrin, where the crosslinked amine polymer is in the form of a base or a hydrochloride or carbonate salt and particles that are chemically coated with polycarbophil using EDC as a coupling agent and where the polycarbophil coated crosslinked amine polymer has: an in vitro competitive phosphate binding capacity that is 2 to 10 times that of uncoated epichlorohydrin crosslinked polyallylamine hydrochloride, a change in in vitro competitive phosphate binding capacity between 1 and 5 hours of less than 50%, an in vitro competitive phosphate binding capacity at 1 hour of greater than 0.5 mmol/g, an in vitro competitive phosphate binding capacity at 5 hours of greater than 0.5 mmol/g, a coating that has a coating stability at pH 1 such that less than 50% of said coating is lost, a coating that has a coating stability at pH 4 such that less than 30% of said coating is lost and/or a coating that has a coating stability in the presence of counterions wherein 70% or less of said coating is lost.

In some embodiments, the polycarbophil coated crosslinked amine polymer comprises a repeat unit according to the following formula:

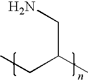

which is crosslinked with 9.8 mol % or 10 mol % epichlorohydrin, where the crosslinked amine polymer is in the form of a base or a hydrochloride or carbonate salt and particles that are ionically coated or chemically coated with polycarbophil, where the coating is thermally treated and where the polycarbophil coated crosslinked amine polymer has: an in vitro competitive phosphate binding capacity that is 2 to 10 times that of uncoated epichlorohydrin crosslinked polyallylamine hydrochloride, a change in in vitro competitive phosphate binding capacity between 1 and 5 hours of less than 50%, an in vitro competitive phosphate binding capacity at 1 hour of greater than 0.5 mmol/g, an in vitro competitive phosphate binding capacity at 5 hours of greater than 0.5 mmol/g, a coating that has a coating stability at pH 1 such that less than 50% of said coating is lost, a coating that has a coating stability at pH 4 such that less than 30% of said coating is lost and/or a coating that has a coating stability in the presence of counterions wherein 70% or less of said coating is lost.

The term “phosphate imbalance disorder” as used herein refers to conditions in which the level of phosphorus present in the body is abnormal. One example of a phosphate imbalance disorder includes hyperphosphatemia. The term “hyperphosphatemia” as used herein refers to a condition in which the element phosphorus is present in the body at an elevated level. Typically, a patient is often diagnosed with hyperphosphatemia if the blood phosphate level is, for example, above about 4.0 or 4.5 milligrams per deciliter of blood, for example above about 5.0 mg/dl, such as above about 5.5 mg/dl, for example above 6.0 mg/dl, and/or a severely impaired glomerular filtration rate such as, for example, less than about 20% of normal. The present invention may also be used to treat patients suffering from hyperphosphatemia in End Stage Renal Disease and who are also receiving dialysis treatment (e.g., hemodialysis or peritoneal dialysis).

Other diseases that can be treated with the methods, polymers, compositions and kits of the present invention include hypocalcemia, hyperparathyroidism, depressed renal synthesis of calcitriol, tetany due to hypocalcemia, renal insufficiency, and ectopic calcification in soft tissues including calcifications in joints, lungs, kidney, conjuctiva, and myocardial tissues. Also, the present invention can be used to treat Chronic Kidney Disease (CKD), End Stage Renal Disease (ESRD) and dialysis patients, including prophylactic treatment of any of the above.

The polycarbophil coated crosslinked amine polymers and compositions described herein can be used as an adjunct to other therapies, e.g., those employing dietary control of phosphorus intake, dialysis, inorganic metal salts and/or other polymer resins.

The compositions of the present invention are also useful in removing chloride, bicarbonate, oxalate, and bile acids from the gastrointestinal tract. Polycarbophil coated crosslinked amine polymers removing oxalate compounds or ions find use in the treatment of oxalate imbalance disorders, such as oxalosis or hyperoxaluria that increases the risk of kidney stone formation. Polycarbophil coated crosslinked amine polymers removing chloride compounds or ions find use in treating acidosis, heartburn, acid reflux disease, sour stomach or gastritis, for example. In some embodiments, the compositions of the present invention are useful for removing fatty acids, bilirubin, and related compounds. Some embodiments may also bind and remove high molecular weight molecules like proteins, nucleic acids, vitamins or cell debris.

The present invention provides methods, pharmaceutical compositions, and kits for the treatment of animals. The term “animal” or “animal subject” or “patient” as used herein includes humans as well as other mammals (e.g., in veterinary treatments, such as in the treatment of dogs or cats, or livestock animals such as pigs, goats, cows or horses) or other livestock such as chickens and the like. One embodiment of the invention is a method of removing phosphorous-containing compounds such as organophosphates or phosphate from the gastrointestinal tract, such as the stomach, small intestine or large intestine of an animal by administering an effective amount of at least one of the polycarbophil coated crosslinked amine polymers described herein.

The term “treating” and its grammatical equivalents as used herein include achieving a therapeutic benefit and/or a prophylactic benefit. By therapeutic benefit is meant eradication, amelioration, or prevention of the underlying disorder being treated. For example, in a hyperphosphatemia patient, therapeutic benefit includes eradication or amelioration of the underlying hyperphosphatemia. Also, a therapeutic benefit is achieved with the eradication, amelioration, or prevention of one or more of the physiological symptoms associated with the underlying disorder such that an improvement is observed in the patient, notwithstanding that the patient may still be afflicted with the underlying disorder. For example, administration of polycarbophil coated crosslinked amine polymers, described herein, to a patient suffering from renal insufficiency and/or hyperphosphatemia provides therapeutic benefit not only when the patient's serum phosphate level is decreased, but also when an improvement is observed in the patient with respect to other disorders that accompany renal failure and/or hyperphosphatemia like ectopic calcification and renal osteodistrophy. For prophylactic benefit, for example, the polycarbophil coated crosslinked amine polymers may be administered to a patient at risk of developing hyperphosphatemia or to a patient reporting one or more of the physiological symptoms of hyperphosphatemia, even though a diagnosis of hyperphosphatemia may not have been made.

The compositions may also be used to control serum phosphate in subjects with elevated phosphate levels, for example, by changing the serum level of phosphate towards a normal or near normal level, for example, towards a level that is within 10% of the normal level of a healthy patient.

Other embodiments of the invention are directed towards pharmaceutical compositions comprising at least one of the polycarbophil coated crosslinked amine polymers or a pharmaceutically acceptable salt of the polycarbophil coated crosslinked amine polymer, and one or more pharmaceutically acceptable excipients, diluents, or carriers and optionally additional therapeutic agents. The compositions may be lyophilized or dried under vacuum or oven before formulating.

The excipients or carriers are “acceptable” in the sense of being compatible with the other ingredients of the formulation and not deleterious to the recipient thereof. The formulations can conveniently be presented in unit dosage form and can be prepared by any suitable method. The methods typically include the step of bringing into association the agent with the excipients or carriers such as by uniformly and intimately bringing into association the crosslinked amine polymer with the excipients or carriers and then, if necessary, dividing the product into unit dosages thereof.

The pharmaceutical compositions of the present invention include compositions wherein the polycarbophil coated crosslinked amine polymers are present in an effective amount, i.e., in an amount effective to achieve therapeutic and/or prophylactic benefit. The actual amount effective for a particular application will depend on the patient (e.g., age, weight, etc.) the condition being treated; and the route of administration.

The dosages of the polycarbophil coated crosslinked amine polymers in animals will depend on the disease being, treated, the route of administration, and the physical characteristics of the animal being treated. Such dosage levels in some embodiments for either therapeutic and/or prophylactic uses may be from about 1 gm/day to about 30 gm/day, for example from about 2 gm/day to about 20 gm/day or from about 3 gm/day to about 7 gm/day. The dose of the polycarbophil coated crosslinked amine polymers described herein can be less than about 50 gm/day, less than about 40 gm/day, less than about 30 gm/day, less than about 20 gm/day, and less than about 10 gm/day.

Typically, the polycarbophil coated crosslinked amine polymers can be administered before or after a meal, or with a meal. As used herein, “before” or “after” a meal is typically within two hours, preferably within one hour, more preferably within thirty minutes, most preferably within ten minutes of commencing or finishing a meal, respectively.

Generally, it is preferred that the polycarbophil coated crosslinked amine polymers are administered along with meals. The polycarbophil coated crosslinked amine polymers may be administered one time a day, two times a day, or three times a day. Preferably the polycarbophil coated crosslinked amine polymers are administered once a day with the largest meal.

Preferably, the polycarbophil coated crosslinked amine polymers may be used for therapeutic and/or prophylactic benefits and can be administered alone or in the form of a pharmaceutical composition. The pharmaceutical compositions comprise the polycarbophil coated crosslinked amine polymers, one or more pharmaceutically acceptable carriers, diluents or excipients, and optionally additional therapeutic agents. For example, the polycarbophil coated crosslinked amine polymers of the present invention may be co-administered with other active pharmaceutical agents depending on the condition being treated. Examples of pharmaceutical agents that may be co-administered include, but are not limited to:

Other phosphate sequestrants including pharmaceutically acceptable lanthanum, calcium, aluminum, magnesium, iron and zinc compounds, such as acetates, carbonates, oxides, hydroxides, citrates, alginates, and ketoacids thereof;

Calcium compounds, including calcium carbonate, acetate (such as PhosLo® calcium acetate tablets), citrate, alginate, and ketoacids;

Aluminium-based phosphate sequestrants, such as Amphojel® aluminium hydroxide gel; and

Lanthanide compounds such as lanthanum carbonate (Fosrenol®).

Other phosphate sequestrants suitable for co-administration include pharmaceutically acceptable magnesium compounds. Various examples of pharmaceutically acceptable magnesium compounds are described in U.S. Provisional Application No. 60/734,593 filed Nov. 8, 2005, the entire teachings of which are incorporated herein by reference. Specific suitable examples include magnesium oxide, magnesium hydroxide, magnesium halides (e.g., magnesium fluoride, magnesium chloride, magnesium bromide and magnesium iodide), magnesium alkoxides (e.g., magnesium ethoxide and magnesium isopropoxide), magnesium carbonate, magnesium bicarbonate, magnesium formate, magnesium acetate, magnesium trisilicates, magnesium salts of organic acids, such as fumaric acid, maleic acid, acrylic acid, methacrylic acid, itaconic acid and styrenesulfonic acid, and a combination thereof.

Other phosphate sequestrants suitable for co-administration include various examples of pharmaceutically acceptable zinc compounds which are described in PCT Application No. PCT/US2005/047582 filed Dec. 29, 2005, the entire teachings of which are incorporated herein by reference. Specific suitable examples of pharmaceutically acceptable zinc compounds include zinc acetate, zinc bromide, zinc caprylate, zinc carbonate, zinc chloride, zinc citrate, zinc formate, zinc hexafluorosilicate, zinc iodate, zinc iodide, zinc iodide-starch, zinc lactate, zinc nitrate, zinc oleate, zinc oxalate, zinc oxide, calamine (zinc oxide with a small proportion of ferric oxide), zinc p-phenolsulfonate, zinc propionate, zinc salicylate, zinc silicate, zinc stearate, zinc sulfate, zinc sulfide, zinc tannate, zinc tartrate, zinc valerate and zinc ethylenebis(dithiocarbamate). Another example includes poly(zinc acrylate).

When referring to any of the above-mentioned phosphate sequestrants, it is to be understood that mixtures, polymorphs and solvates thereof are encompassed.

In some embodiments, a mixture of the phosphate sequestrants described above can be used in combination with pharmaceutically acceptable ferric or ferrous iron salts.

In other embodiments, the phosphate sequestrant used in combination with polycarbophil coated crosslinked amine polymers of the present invention is not a pharmaceutically acceptable magnesium compound. In yet other embodiments, the phosphate sequestrant used in combination with the pharmaceutically acceptable polycarbophil coated crosslinked amine polymers is not a pharmaceutically acceptable zinc compound.

The invention also includes methods and pharmaceutical compositions directed to a combination therapy of the polycarbophil coated crosslinked amine polymers in combination with a phosphate transport inhibitor or an alkaline phosphatase inhibitor. Alternatively, a mixture of the polycarbophil coated crosslinked amine polymers may be employed together with a phosphate transport inhibitor or an alkaline phosphatase inhibitor.

Suitable examples of such phosphate transport inhibitors can be found in co-pending U.S. Application Publication Nos. 2004/0019113 and 2004/0019020 and WO 2004/085448, the entire teachings of each of which are incorporated herein by reference.

A large variety of organic and inorganic molecules are inhibitors to alkaline phosphatase (ALP) (see, for example, U.S. Pat. No. 5,948,630, the entire teachings of which are incorporated herein by reference) and may be co-administered with the polycarbophil coated crosslinked amine polymer. Examples of such alkaline phosphatase inhibitors include orthophosphate, arsenate, L-phenylalanine, L-homoarginine, tetramisole, levamisole, L-p-Bromotetramisole, 5,6-Dihydro-6-(2-naphthyl)imidazo-[2,1-b]thiazole (napthyl) and derivatives thereof. The preferred inhibitors include, but are not limited to, levamisole, bromotetramisole, and 5,6-Dihydro-6-(2-naphthyl)imidazo-[2,1-b]thiazole and derivatives thereof.

This co-administration can include simultaneous administration of the two agents in the same dosage form, simultaneous administration in separate dosage forms, and separate administration. For example, for the treatment of hyperphosphatemia, the polycarbophil coated crosslinked amine polymers may be co-administered with calcium salts which are used to treat hypocalcemia resulting from hyperphosphatemia.

The pharmaceutical compositions of the invention can be formulated as a tablet, sachet, slurry, food formulation, troche, capsule, elixir, suspension, syrup, wafer, chewing gum or lozenge.

Preferably, the polycarbophil coated crosslinked amine polymers or the pharmaceutical compositions comprising the polycarbophil coated crosslinked amine polymers are administered orally. Illustrative of suitable methods, vehicles, excipients and carriers are those described, for example, in Remington's Pharmaceutical Sciences, 19th ed., the contents of which is incorporated herein by reference.

Pharmaceutical compositions for use in accordance with the present invention may be formulated in conventional manner using one or more physiologically acceptable carriers comprising excipients and auxiliaries which facilitate processing of the active polycarbophil coated crosslinked amine polymers into preparations which can be used pharmaceutically. Proper formulation is dependent upon the route of administration chosen. Suitable techniques for preparing pharmaceutical compositions of the polycarbophil coated crosslinked amines are well known in the art.

In some aspects of the invention, the polycarbophil coated crosslinked amine polymer(s) provide mechanical and thermal properties that are usually performed by excipients, thus decreasing the amount of such excipients required for the formulation. In some embodiments the polycarbophil coated crosslinked amine polymer or composition constitutes over about 30 wt. %, for example over about 40 wt. %, over about 50 wt. %, preferably over about 60 wt. %, over about 70 wt. %, more preferably over about 80 wt. %, over about 85 wt. %, over about 90 wt. %, over about 95 wt. % or over about 99 wt. % of the composition, the remainder comprising suitable excipient(s).

In some embodiments, the compressibility of tablets is strongly dependent upon the degree of hydration (moisture content) of the polycarbophil coated crosslinked amine polymer. Preferably, the polycarbophil coated crosslinked amine polymer has a moisture content of about 5% by weight or greater, more preferably, the moisture content is from about 5% to about 9% by weight, and most preferably about 7% by weight. It is to be understood that in embodiments in which the polycarbophil coated crosslinked amine polymer is hydrated, the water of hydration is considered to be a component of the polycarbophil coated crosslinked amine polymer.

The tablet can further comprise one or more excipients, such as hardeners, glidants and lubricants, which are well known in the art. Suitable excipients include colloidal silicon dioxide, stearic acid, magnesium silicate, calcium silicate, sucrose, calcium stearate, glyceryl behenate, magnesium stearate, talc, zinc stearate and sodium stearylfumarate.

The tablet core of embodiments of the invention may be prepared by a method comprising the steps of: (1) hydrating or drying the polycarbophil coated crosslinked amine polymer to the desired moisture level; (2) blending the polycarbophil coated crosslinked amine polymer with any excipients; and (3) compressing the blend using conventional tableting technology.

In some embodiments, the invention relates to a stable, swallowable coated tablet, particularly a tablet comprising a hydrophilic core, such as a tablet comprising the polycarbophil coated crosslinked amine polymer, as described above. In one embodiment, the coating composition comprises a cellulose derivative and a plasticizing agent. The cellulose derivative is, preferably, hydroxypropylmethylcellulose (HPMC). The cellulose derivative can be present as an aqueous solution. Suitable hydroxypropylmethylcellulose solutions include those containing HPMC low viscosity and/or HPMC high viscosity. Additional suitable cellulose derivatives include cellulose ethers useful in film coating formulations. The plasticizing agent can be, for example, an acetylated monoglyceride such as diacetylated monoglyceride. The coating composition can further include a pigment selected to provide a tablet coating of the desired color. For example, to produce a white coating, a white pigment can be selected, such as titanium dioxide.

In one embodiment, the coated tablet of the invention can be prepared by a method comprising the step of contacting a tablet core of the invention, as described above, with a coating solution comprising a solvent, at least one coating agent dissolved or suspended in the solvent and, optionally, one or more plasticizing agents. Preferably, the solvent is an aqueous solvent, such as water or an aqueous buffer, or a mixed aqueous/organic solvent. Preferred coating agents include cellulose derivatives, such as hydroxypropylmethylcellulose. Typically, the tablet core is contacted with the coating solution until the weight of the tablet core has increased by an amount ranging from about 4% to about 6%, indicating the deposition of a suitable coating on the tablet core to form a coated tablet.

Other pharmaceutical excipients useful in some compositions of the invention include a binder, such as microcrystalline cellulose, carbopol, providone and xanthan gum; a flavoring agent, such as mannitol, xylitol, maltodextrin, fructose, or sorbitol; a lubricant, such as vegetable based fatty acids; and, optionally, a disintegrant, such as croscarmellose sodium, gellan gum, low-substituted hydroxypropyl ether of cellulose, sodium starch glycolate. Such additives and other suitable ingredients are well-known in the art; see, e.g., Gennaro A R (Ed), Remington's Pharmaceutical Sciences, 19th Edition.

In some embodiments the polycarbophil coated crosslinked amine polymers of the invention are provided as pharmaceutical compositions in the form of chewable tablets. In addition to the active ingredient, the following types of excipients are commonly used: a sweetening agent to provide the necessary palatability, plus a binder where the former is inadequate in providing sufficient tablet hardness; a lubricant to minimize frictional effects at the die wall and facilitate tablet ejection; and, in some formulations a small amount of a disintegrant is added to facilitate mastication. In some embodiments the invention provides a pharmaceutical composition formulated as a chewable tablet, comprising a polycarbophil coated crosslinked amine polymer described herein, a filler, and a lubricant. In some embodiments the invention provides a pharmaceutical composition formulated as a chewable tablet, comprising a polycarbophil coated crosslinked amine polymer described herein, a filler, and a lubricant, wherein the filler is chosen from the group consisting of sucrose, mannitol, xylitol, maltodextrin, fructose, and sorbitol, and wherein the lubricant is a magnesium fatty acid salt, such as magnesium stearate.

In one embodiment, the polycarbophil coated crosslinked amine polymer is pre-formulated with a high Tg/high melting point low molecular weight excipient such as mannitol, sorbose, sucralose, saccharin or sodium saccharin, cyclamates, aspartame or sucrose in order to form a solid solution wherein the polymer and the excipient are intimately mixed. Methods of mixing such as extrusion, spray-drying, chill drying, lyophilization, or wet granulation are useful. Indication of the level of mixing is given by known physical methods such as differential scanning calorimetry or dynamic mechanical analysis.

In some embodiments the polycarbophil coated crosslinked amine polymers of the invention are provided as pharmaceutical compositions in the form of liquid formulations. In some embodiments the pharmaceutical composition contains polymer dispersed in a suitable liquid excipient. Suitable liquid excipients are known in the art; see, e.g., Remington's Pharmaceutical Sciences.

All publications and patent applications mentioned in this specification are herein incorporated by reference to the same extent as if each individual publication or patent application was specifically and individually indicated to be incorporated by reference.

It will be apparent to one of ordinary skill in the art that many changes and modification can be made to the disclosures presented herein without departing from the spirit or scope of the appended claims.

### EXAMPLES

### Example 1

### Preparation of Compound I

3 g of 9.8 mol % epichlorohydrin-crosslinked polyallylamine hydrochloride was dispersed in 200 ml of deionized water and stirred for 1 hour. 90 mg of polycarbophil was added to this dispersion and the mixture was stirred for 15 minutes. The solids were filtered from the solution, washed with 300 ml deionized water and vacuum dried for 48 hours to yield the desired product.

### Example 2

### Preparation of Compound II

Polycarbophil-coated epichlorohydrin-crosslinked polyallylamine hydrochloride was prepared according to Example 1 and then was placed in a vacuum oven at 90° C. The oven was purged of air using three cycles of nitrogen gas and vacuum. The thermal treatment was continued for 14 hours.

### Example 3

### Preparation of Compound III

3 g of 9.8 mol % epichlorohydrin-crosslinked polyallylamine hydrochloride was dispersed in 200 ml of deionized water and stirred for 1 hour. In a separate container, 90 mg of polycarbophil was dissolved in 50 ml of deionized water. 0.09585 g of N-(3-dimethylaminopropyl)-N′-ethylcarbodiimide hydrochloride (EDC) was added to the polycarbophil solution and the solution was stirred for 20 minutes. The polycarbophil solution was combined with the polyallylamine dispersion and stirred for 15 minutes. The solids were filtered, washed with 300 ml of deionized water and dried in a freeze dryer for 48 hours.

The coating stability, in vitro non-completive phosphate binding and the in vitro competitive phosphate binding capacities for each of Compounds I, II and III were evaluated according to the procedures in the test methods. The results are presented below:

TABLE 1
Polycarbophil Coating Stability at Various
pHs and in the Presence of a Counterion

| | | Coating loss | | | | |
|---|---|---|---|---|---|---|
| | | % due to | Coating loss % due to pH | | | |
| Compound | Binding % | counterion | pH 1 | pH 4 | pH 6 | pH 8 |
| Compound I | 1.9 | 60 | 41 | 21 | 22 | 20 |
| Compound II | 2.1 | 6.6 | 17 | 0.0 | 7.0 | 6.0 |
| Compound III | 2.3 | 0.6 | 10 | 6.8 | 5.8 | 4.1 |

TABLE 2
In vitro Non-Competitive Phosphate Binding Capacity

Phosphate Binding
Capacity (mmol/g)

| Compound | 1 hour | 5 hours |
|---|---|---|
| Uncoated sevelamer | 5.0 | 5.0 |
| hydrochloride | | |

TABLE 3
In vitro Competitive Phosphate Binding Capacity

Phosphate Binding
Capacity (mmol/g)

| Compound | 1 hour | 5 hours |
|---|---|---|
| Compound I | 4.5 | 4.1 |
| Compound II | 4.4 | 3.6 |
| Compound III | 3.0 | 1.0 |
| Uncoated sevelamer | 0.69 | 0.39 |
| hydrochloride | | |

### Test Methods

### Binding % and Coating Stability

Polycarbophil-coated polyallylamine was prepared according to the procedures described in the Examples. The % binding was determined by analyzing the filtrate from the filtration step for each preparation using a UV spectrophotometer at a wavelength of 445 nm. The absorbance of the filtrate was evaluated, the concentration of the polycarbophil in the filtrate was determined according to Beer-Lambert's law and the % binding was determined according to the following equation:

$$Binding \% = 1 - \frac{( {m}_{PCPi} - {m}_{PCPf} )}{{m}_{PCPi}}$$

where m_{PCPi}=the mass of polycarbophil added to the solution; and
m_{PCPf}=the mass of the polycarbophil in the filtrate.

The coated polyallylamine was then divided into 5 equal aliquots (A-E) and subjected to the following coating stability studies:

Aliquot A: Aliquot A was combined with 25 ml of deionized water with stirring, the pH was adjusted to 1.0 using concentrated HCl and the solution was stirred for 2 hours and filtered. The filtrate was pH-adjusted to 7.0 using 0.1 N Phosphate buffer, the absorbance of the filtrate was measured at a wavelength of 445 nm using a UV spectrophotometer and the concentration of polycarbophil in the filtrate was determined as discussed above for the Binding %. The 0.1 N Phosphate buffer used was prepared as follows: 5.836 g of sodium phosphate dibasic heptahydrate (Na₂HPO₄).7H₂O and 15.466 g of sodium phosphate monobasic hydrate (NaH₂PO₄).H₂O were added to 500 ml of deionized water and placed in a 1 L volumetric flask. The solution was stirred with a magnetic stirrer until all solids dissolved and diluted to 1 L by with deionized water. The coating loss % due to pH was then determined according to the following equation:

$$Coating Loss \% due to pH 1 = \frac{( {m}_{PCPC} - {m}_{pH 1} )}{{m}_{PCPC}}$$

where m_{PCPC}=(m_{PCPi}−m_{PCPf}); and
m_{pH 1}=the mass of polycarbophil in the filtrate for Aliquot A.

Aliquot B: Aliquot B was combined with 25 ml of deionized water with stirring, the pH was adjusted to 4.0 using concentrated HCl and the solution was stirred for 2 hours and filtered. The filtrate was pH-adjusted to 7.0 using 0.1 N Phosphate buffer prepared as discussed above with respect to Aliquot A, the absorbance of the filtrate was measured at a wavelength of 445 nm using a UV spectrophotometer and the concentration of polycarbophil in the filtrate was determined as discussed above for the Binding %. The coating loss % due to pH was determined according to the following equation:

$$Coating Loss \% due to pH 4 = \frac{( {m}_{PCPC} - {m}_{pH 4} )}{{m}_{PCPC}}$$

where m_{PCPC}=(m_{PCPi}−m_{PCPf}); and

m_{pH 4}=the mass of polycarbophil in the filtrate for Aliquot B.

Aliquot C: Aliquot C was combined with 25 ml of deionized water with stirring, the pH was adjusted to 6.0 using 0.1 N NaOH and the solution was stirred for 2 hours and filtered. The filtrate was pH-adjusted to 7.0 using 0.1 N Phosphate buffer prepared as discussed above with respect to Aliquot A, the absorbance of the filtrate was measured at a wavelength of 445 nm using a UV spectrophotometer and the concentration of polycarbophil in the filtrate was determined as discussed above for the Binding %. The coating loss % due to pH was determined according to the following equation:

$$Coating Loss \% due to pH 6 = \frac{( {m}_{PCPC} - {m}_{pH 6} )}{{m}_{PCPC}}$$

where m_{PCPC}=(m_{PCPi}−m_{PCPf}); and

m_{pH 6}=the mass of polycarbophil in the filtrate for Aliquot C.

Aliquot D: Aliquot D was combined with 25 ml of deionized water with stirring, the pH was adjusted to 8.0 using 0.1 N NaOH and the solution was stirred for 2 hours and filtered. The filtrate was pH-adjusted to 7.0 using 0.1 N Phosphate buffer prepared as discussed above with respect to Aliquot A, the absorbance of the filtrate was measured at a wavelength of 445 nm using a UV spectrophotometer and the concentration of polycarbophil in the filtrate was determined as discussed above for the Binding %. The coating loss % due to pH was determined according to the following equation:

$$Coating Loss \% due to pH 8 = \frac{( {m}_{PCPC} - {m}_{pH 8} )}{{m}_{PCPC}}$$

where m_{PCPC}=(M_{PCPi}−m_{PCPf}); and

m_{pH 8}=the mass of polycarbophil in the filtrate for Aliquot D.

Aliquot E: Aliquot E was combined with 100 ml of deionized water and 80.82 mg of poly(diallyldimethylammonium hydrochloride) with stirring, the solution was stirred for 15 minutes and filtered. The filtrate was pH-adjusted to 7.0 using 0.1 N Phosphate buffer prepared as discussed above with respect to Aliquot A, the absorbance of the filtrate was measured at a wavelength of 445 nm using a UV spectrophotometer and the concentration of polycarbophil in the filtrate was determined as discussed above for the Binding %. The Coating loss % due to counterion was determined according to the following equation:

$$Coating Loss \% due to counterion = \frac{( {m}_{PCPC} - {m}_{pH 8} )}{{m}_{PCPC}}$$

where m_{PCPC}=(m_{PCPi}−m_{PCPf}); and

m_{pH 8}=the mass of polycarbophil in the filtrate for Aliquot E.

### Non-Competitive Phosphate Binding Capacity

### Buffer Preparation:

0.680 g of KH₂PO₄, 10.663 g of (N,N-Bis-2-Hydroxyethyl)-2-Aminomethanesulfonic Acid (BES) and 2.338 g of NaCl were weighed into a 500 ml volumetric flask. 300 ml of deionized water was added and the solids were dissolved. Additional deionized water was added until the total volume of buffer was 500 ml. The pH was adjusted to 7.0 using 1 N NaOH.

### Sample Preparation:

The loss on drying by TGA (% LOD) of 25 mg of each coated polymer was determined on a Thermogravimetric Analyzer, TA Instruments, Model TGA Q 500, purged with nitrogen and using platinum pans. The following heating conditions were used:

Heating rate: 10° C./min

End temperature: 85° C.

Hold time: 60 minutes

The % LOD was determined as the % weight loss over 65 minutes and the result was used to calculate the target sample weight with the following the formula:

Weight=33.35 mg/(1−(LOD/100))

### Binding Procedure:

The calculated target sample weight per coated polymer was weighed into each of two 50 ml plastic sample bottles. A 25 ml aliquot of the 10 mM Phosphate Buffer Solution was transferred into each of the sample bottles. The solutions were mixed well by vortexing and then shaken in an orbital shaker at 37° C. and 250 RPMs for 60 minutes. During shaking it was ensured that the polymer particles did not adhere to the walls or lid of the sample bottle. After 60 minutes the shaker was stopped and the polymer was allowed to settle. An aliquot of exactly 2.0 ml was taken from each solution. The aliquots were filtered into small vials using a disposable syringe and 25 mm syringe filter and then diluted at a ratio of 1 part solution to 9 parts DI water. The sample bottles were shaken for a further 4 hours (total of 5 hours altogether) and the sampling procedure was repeated. Four phosphate standards were prepared by diluting the 10 mM Phosphate Buffer Solution as follows:

| Std Conc, | Volume of 10 mM | Volume of H₂O, | |
|---|---|---|---|
| mM | Phosphate Solution, ml | ml | Total Volume, ml |
| 0.30 | 0.75 | 24.25 | 25 |
| 0.50 | 0.50 | 9.50 | 10 |
| 0.75 | 0.75 | 9.25 | 10 |
| 1.00 | 1.00 | 9.00 | 10 |

The standards and samples were analyzed by ion chromatography using a Dionex ICS3000 instrument with conductivity detection. The 0.75 mM Standard was used as a check standard to verify the system suitability by re-injecting this standard after every 6 sample injections. The following instrument conditions were used:

Column: Dionex, AS11-HC, 4×250 mm,

Guard Column: AG 11-HC, 4×50 mm,

Mobile Phase=40 mM KOH (using eluent generator)

Conductivity detector current set at 149 mA

Column Temperature: 35° C.

Flow rate: 1.5 mL/min

Injection volume: 25 μl

Run time: 6 minutes

Retention time of phosphate: ˜4 mins

A standard curve was prepared and the unbound phosphate (mM) for each test solution was calculated taking into account the 10-fold dilution. The bound phosphate was determined using the following equation:

Bound PO₄(mmol/g)=[(10−Unbound PO₄)×Vol.×1000]/MassP

where: Vol.=volume of test solution (L)

MassP=LOD adjusted mass of polymer (mg)

The results from the duplicate analyses were averaged.

### Competitive Phosphate Binding Capacity

### Buffer Preparation:

0.680 g of KH₂PO₄, 10.663 g (N,N-Bis-2-Hydroxyethyl)-2-Aminomethanesulfonic Acid (BES) and 2.338 g of NaCl were weighed into a 500 ml volumetric flask. 300 ml of deionized water was added and the solids were dissolved. Additional deionized water was added until the total volume of buffer was 500 ml. A 10 mL aliquot of this solution was taken and stored for use in the preparation of standards. 3.537 g of Glycochenodeoxycholic acid, sodium salt (GCDC) and 2.285 g of oleic acid (OA), sodium salt were added to the remaining 490 ml of buffer solution and the pH was adjusted to pH 7.0 with 1 N HCl. The solution was well mixed. (Note that OA did not dissolve but formed a suspension. It was ensured that the solution was well mixed and the suspended OA was mixed as homogenously as possible before taking aliquots.)

### Sample Preparation:

The loss on drying by thermogravimetric analysis (% LOD) of 25 mg of each polymer was determined on a Thermogravimetric Analyzer, TA Instruments, Model TGA Q 500, purged with nitrogen and using platinum pans. The following heating conditions were used:

Heating rate: 10° C./min

End temperature: 85° C.

Hold time: 60 minutes

The % LOD was determined as the % weight loss over 65 minutes and the result was used to calculate the target sample weight with the following the formula:

Weight=33.35 mg/(1−(LOD/100))

### Binding Procedure:

The calculated target sample weight per polymer was weighed into each of two 50 ml plastic sample bottles. A 25 ml aliquot of the 10 mM Phosphate Buffer Solution with Acids was transferred into each of the sample bottles. The solutions were mixed well by vortexing and then shaken in an orbital shaker at 37° C. and 250 RPMs until each time point for sampling. During shaking it was ensured that the polymer particles did not adhere to the walls or lid of the sample bottle. At each time point, the shaker was stopped and the polymer was allowed to settle. An aliquot of exactly 2.0 ml was taken from each solution. The aliquots were filtered into small vials using a disposable syringe and 25 mm syringe filter and then diluted at a ratio of 1 part solution to 9 parts DI water. The sample bottles were shaken and the sampling procedure was repeated at each time point. Four phosphate standards were prepared by diluting the 10 mM Phosphate Buffer Solution with Acids as follows:

| Std Conc, | Volume of 10 mM | Volume of H2O, | |
|---|---|---|---|
| mM | Phosphate Solution, ml | ml | Total Volume, ml |
| 0.30 | 0.75 | 24.25 | 25 |
| 0.50 | 0.50 | 9.50 | 10 |
| 0.75 | 0.75 | 9.25 | 10 |
| 1.00 | 1.00 | 9.00 | 10 |

The standards and samples were analyzed by ion chromatography using a Dionex ICS3000 instrument with conductivity detection. The 0.75 mM Standard was used as a check standard to verify the system suitability by re-injecting this standard after every 6 sample injections. The following instrument conditions were used:

Column: Dionex, AS11-HC, 4×250 mm, P/N 052960

Guard Column: AG11-HC, 4×50 mm, P/N 052962

Mobile Phase=40 mM KOH (using eluent generator)

Conductivity detector, current set at 149 mA

Column Temperature: 35° C.

Flow rate: 1.5 mL/min

Injection volume: 25 μl

Run time: 6 mins

Retention time of Phosphate: ˜4 mins

A standard curve was prepared and the unbound phosphate (mM) for each test solution was calculated taking into account the 10-fold dilution. The bound phosphate was determined using the following equation:

Bound PO₄(mmol/g)=[(10−Unbound PO₄)×Vol.×1000]/MassP

where: Vol.=volume of test solution (L)
MassP=LOD adjusted mass of polymer (mg)

The results from the duplicate analyses were averaged.

### Crosslinked Amine Polymer Urinary Phosphorous Reduction (In Vivo-Rats)

House male Sprague Dawley (SD) rats may be used for the experiments. The rats are placed singly in wire-bottom cages, fed with Purina 5002 diet, and allowed to acclimate for at least 5 days prior to experimental use.

To establish baseline phosphorus excretion, the rats are placed in metabolic cages for 48 hours. Their urine is collected and its phosphorus content analyzed with a Hitachi analyzer to determine phosphorus excretion in mg/day. Any rats with outlying values should be excluded; and the remainder of the rats is distributed into groups.

Purina 5002 may be used as the standard diet. The crosslinked amine polymer being tested is mixed with Purina 5002 to result in a final crosslinked amine polymer concentration of 0.25% by weight of the feed. Cellulose at 0.5% by weight is used as a negative control. Sevelamer at 0.5% by weight is used as a positive control. For each rat, 200 g of diet is prepared.

Each rat is weighed and placed on the standard diet. After 4 days the standard diet is replaced with the treatment diet (or control diet for the control group). On days 5 and 6, urine samples from the rats at 24 hours (+/−30 minutes) are collected and analyzed. The test rats are again weighed, and any weight loss or gain is calculated. Any remaining food is also weighed to calculate the amount of food consumed per day. A change in phosphorus excretion relative to baseline and cellulose negative control is calculated. Percentage reduction of urinary phosphorous may be determined by the following equation:

% Reduction of Urinary Phosphorous=[(urinary phosphorous of negative control(mg/day)−urinary phosphorous of experimental(mg/day))/urinary phosphorous of negative control(mg/day)]×100.

In-Process Swelling Ratio (ml/g)

The in-process swelling ratio (SR) of polymers may be determined using the following equation:

SR=(weight of wet gel(g)−weight of dry polymer(g))/weight of dry polymer(g).

### Bile Acid Binding Capacity

After analyzing the competitive phosphate binding of a polymer sample by ion chromatography the bile acid binding capacity of the same samples may be analyzed using HPLC according to the following procedure.

### Buffer Preparation:

0.177 g of GCDC may be weighed into a 25 ml volumetric flask and diluted to the mark using a 100 mM morpholinoethane sulfonic acid stock solution to form a 15 mM GCDC stock solution. Four standards having the following concentrations may be prepared by diluting the GCDC stock solution in volumetric flasks as follows:

| Standard Conc | Volume 15 mM | Vol. Flask |
|---|---|---|
| (mM) | GCDC stock (μL) | (mL) |
| 1.50 | 1000 | 10 |
| 1.00 | 750 | 10 |
| 0.75 | 500 | 10 |
| 0.48 | 800 | 25 |

A blank may be prepared by diluting the MES buffer stock 1-to-10. For the HPLC determination, the following parameters may be used:
Column: Platinum EPS-C18, 33×7 mm, 3 micron, rocket format
MP: A=15 mM ammonium acetate, pH 5.30 (adjust pH with an 8/2 by volume acetic acid/acetonitrile solution)
MP B=acetonitrile
Flow rate: 2 ml/min
Column Temp: 30° C.
Injection Volume: 10 μl
UV Detection: 210 nm

Using the following gradient:

| Time (minutes) | % B |
|---|---|
| 0 | 20 |
| 2 | 20 |
| 4 | 95 |

with stop run=4.0 minutes and post run=2.5 minutes.

The following injection format may be used: Blank twice, Standards twice, Blank, then test samples once each with the 1.0 mM standard injected after every 9 sample injections for system suitability testing. The system is suitable if the difference between the original standards and the suitability standard is less than 5%.

A standard curve may be set up and the unbound GCDC (mM) for each test solution can be calculated. The bound GCDC may then be determined using the following equation:

Bound GCDC(mmol/g)=[(15−Unbound GCDC)×Vol.×1000]/MassP

where: Vol.=volume of test solution (L) and
MassP=LOD adjusted mass of polymer (mg)

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A pharmaceutical composition comprising
a. coated polymer particles, said particles comprising crosslinked particles of polyallylamine or a salt thereof coated with a coating comprising polycarbophil; and
b. a pharmaceutically acceptable excipient.

2. (canceled)

3. The composition according to claim 1, wherein said coating further comprises N-(3-dimethylaminopropyl)-N′-ethylcarbodiimide hydrochloride.

4. The composition according to claim 3, wherein said coating comprises a thermally-treated coating.

5. The composition according to claim 3, wherein said coating comprises an ionically coupled coating.

6.-27. (canceled)

28. A pharmaceutical composition comprising a coated crosslinked amine polymer, wherein said crosslinked amine polymer includes repeat units represented by Formula I: 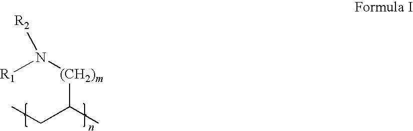
or a copolymer thereof, wherein m is an integer from 0 to 2, n is an integer and each R₁ and each R₂ independently represent hydrogen; substituted or unsubstituted, branched or unbranched C₁-C₆ alkyl; or substituted or unsubstituted, branched or unbranched C₁-C₆ alkylamino, and wherein said crosslinked amine polymer is coated with a coating comprising polycarbophil.

29. The composition according to claim 28, wherein said coating further comprises N-(3-dimethylaminopropyl)-N′-ethylcarbodiimide hydrochloride.

30. The composition according to claim 28, wherein said coating comprises a thermally-treated coating.

31. The composition according to claim 28, wherein said coating comprises an ionically coupled coating.

32. The pharmaceutical composition of claim 28, wherein said crosslinked amine polymer comprises repeat units represented by Formula Ia 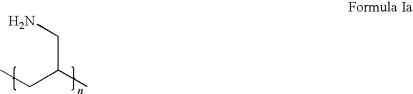
or a copolymer thereof, wherein n is an integer.

33. The composition according to claim 32, wherein said coating further comprises N-(3-dimethylaminopropyl)-N′-ethylcarbodiimide hydrochloride.

34. The composition according to claim 32, wherein said coating comprises a thermally-treated coating.

35. The composition according to claim 32, wherein said coating comprises an ionically coupled coating.

36. The pharmaceutical composition of claim 28, wherein said crosslinked amine polymer comprises repeat units represented by Formula Ib 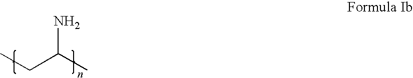
or a copolymer thereof, wherein n is an integer.

37. The composition according to claim 36, wherein said coating further comprises N-(3-dimethylaminopropyl)-N′-ethylcarbodiimide hydrochloride.

38. The composition according to claim 36, wherein said coating comprises a thermally-treated coating.

39. The composition according to claim 36, wherein said coating comprises an ionically coupled coating.

40. A pharmaceutical composition comprising a coated crosslinked amine polymer, wherein said crosslinked amine polymer is sevelamer, and wherein said crosslinked amine polymer is coated with a coating comprising polycarbophil.

41. The pharmaceutical composition of 40 wherein said crosslinked amine polymer is sevelamer hydrochloride.

42. The pharmaceutical composition of 40 wherein said crosslinked amine polymer is sevelamer carbonate.

43. The composition according to claim 41, wherein said coating further comprises N-(3-dimethylaminopropyl)-N′-ethylcarbodiimide hydrochloride.

44. The composition according to claim 42, wherein said coating further comprises N-(3-dimethylaminopropyl)-N′-ethylcarbodiimide hydrochloride.
